(19) United States
(12) Patent Application Publication
(10) Pub. No.: US 2025/0027029 A1
(43) Pub. Date: Jan. 23, 2025

(54) APPARATUS AND METHODS FOR FACILITATING USE OF A SENSOR WITH A VESSEL

(71) Applicant: GLOBAL LIFE SCIENCES SOLUTIONS USA LLC, MARLBOROUGH, MA (US)

(72) Inventors: JEFFREY SOCHA, PRINCETON, MA (US); ELIZABETH PATTON, MARLBOROUGH, MA (US); PAUL SEETO, CLINTON, MA (US)

(73) Assignee: GLOBAL LIFE SCIENCES SOLUTIONS USA LLC, MARLBOROUGH, MA (US)

(21) Appl. No.: 18/223,329

(22) Filed: Jul. 18, 2023

(51) Int. Cl. C12M 1/34; C12M 1/00; C12M 3/00; C12M 3/06; G01N 15/06

(52) U.S. Cl. CPC C12M 41/46; C12M 23/14; C12M 23/28; C12M 23/48; C12M 27/16; G01N 15/0656

(57) ABSTRACT

An apparatus for facilitating use of a sensor with a vessel includes a body having upper and lower portions, the body extending across substantially an entire width of the vessel, the body configured for selective placement on a surface of a bioprocessing device, the body including a sensor recess formed in the upper portion of the body, the sensor recess configured to receive a sensor so that the sensor is at least partially recessed within the body. In use, the body is situated between the surface of the bioprocessing device and the vessel, the body allowing for retention of liquid in the vessel in proximity to the sensor, facilitating use of the sensor with low vessel fill levels.

### BACKGROUND

### Technical Field

Embodiments of the invention relate generally to cellular processing, and, more particularly, to apparatus and methods that facilitate the use of a sensor, e.g., a viable cell density (VCD) sensor, with a vessel.

### DISCUSSION OF ART

Cellular processing devices such as bioreactors, mixers, and fermenters are often employed to carry out biochemical and/or biological processes and/or manipulate liquids and other products of such processes. Such devices often include flexible or collapsible single-use disposable bags/vessels that are supported by an outer rigid structure. The vessels are filled with the desired fluid for processing. The fluid within the vessels may require mixing or agitation to prevent settling of particulates at the bottom of the vessel which may be accomplished through the use of, for example, a rocking platform to which a single-use vessel is attached.

Fluids typically introduced into vessels include liquids, such as cell culture media, serum, salt solutions, buffers, and water, as well as gases such as air, oxygen, carbon dioxide, nitrogen, or mixtures thereof. Fluids are added to establish and maintain suitable growth and/or reaction conditions for producing a product from cellular material within the vessel.

As will be appreciated, analysis of cellular material, e.g., cells, within the vessel is important to ensure conditions are optimal for growth and that growth is occurring. In particular, it may be desirable to analyze cellular material during processing, allowing for real-time or near real-time process control. In some instances, analysis occurs via one or more sensors that are welded to/formed in, or selectively attached to, a wall or surface of the vessel.

For example, it is often desirable to assess viable cell density (VCD) which provides a measurement of the total amount of live cells within the cellular material, e.g., cell culture, in the vessel. This may be accomplished via VCD sensors that measure cell permittivity. Such sensors may include a sensor patch that may be formed in or attached to a lower/bottom surface of the vessel, as well as a transducer, which is connected to the sensor patch during operation.

In use, VCD sensors measure cell permittivity within fluid that is located above the sensor, i.e., the sensor patch must be generally immersed in the fluid. Usage of such sensors with rocking platform devices/bioreactors may be problematic, however, when the volume of fluid in the vessel is relatively low. When connected to the transducer, the sensor patch is elevated off the rocking platform forming an island within the vessel interior. With lower volumes, the VCD sensor may be elevated above the fluid level in the vessel, particularly when the rocking platform is tilted or angled away from the sensor patch and fluid has collected at the opposite end of the vessel. As will be appreciated, this may lead to data loss/inaccurate VCD measurements and/or excessive signal loss.

The elevation of the VCD sensor may also necessitate undesirably or impracticably high minimum vessel fill levels to compensate. Such fill levels may require larger bags/vessels and/or rocking platforms/trays than those currently commercially available. Moreover, biologics are of high value, so it is generally desirable to manufacture only what is needed and minimize waste.

Furthermore, known solutions for addressing erroneous sensor measurements due to fluctuating fluid levels involve complex, frequency spectrum-based systems that synchronize sensor measurements with the mixing motion of the bioreactor. Such systems involve specific hardware, e.g., sensors for measuring mixing motion variables such as angle, position change, momentary movement, etc., of the bioreactor, and software for selectively triggering sensor measurements based on the variables, and/or for selecting a recorded value from a sensor based on measured motion.

In order to reduce equipment complexity and costs, increase efficiency, and for overall convenience, a need exists for an apparatus and method that allows existing rocking platform reactors (for a large installed customer base) to be used with VCD equipped vessels without the need to undesirably increase minimum vessel liquid levels, and without the need for complex hardware and/or measurement synchronization software.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, an apparatus for facilitating use of a sensor with a vessel includes a body having upper and lower portions, the body extending across substantially an entire width of the vessel, the body configured for selective placement on a surface of a bioprocessing device, the body including a sensor recess formed in the upper portion of the body, the sensor recess configured to receive a sensor so that the sensor is at least partially recessed within the body. In use, the body is situated between the surface of the bioprocessing device and the vessel, the body allowing for retention of liquid in the vessel in proximity to the sensor, facilitating use of the sensor with low vessel fill levels.

In an embodiment, the surface of the bioprocessing device may be a tray configured for attachment to a platform of the bioprocessing device that is capable of rocking about an axis to facilitate a bioprocessing procedure within the vessel.

In an embodiment, the sensor in the sensor recess may be a first portion of a viable cell density sensor that is configured to selectively engage a second portion of a viable cell density sensor that is fixed to the vessel.

In an embodiment, the vessel is a single-use bioreactor bag.

In an embodiment, the upper portion of the body may include a first angled surface and a second angled surface, the first angled surface including the sensor recess; and, in use, the body is located proximate to a first end of the bioprocessing device surface with the first angled surface facing the first end of the bioprocessing device surface and the second angled surface facing a second end of the bioprocessing device surface, opposite the first end.

In an embodiment, the body may have a substantially triangular profile or cross-section with a substantially obtuse angle between the first and second angled surfaces.

In an embodiment, the first angled surface may have an angle of approximately 15 degrees from the surface of the bioprocessing device.

In an embodiment, the upper portion of the body may include an angled surface, the angled surface including the sensor recess; and, in use, the body is located proximate to a first end of the bioprocessing device surface and the angled surface is facing a second end of the bioprocessing device surface, opposite the first end.

In an embodiment, the body may have a profile or cross section of a substantially right triangle with the longest side of the triangle being the angled surface having the sensor recess.

In an embodiment, the angled surface may be at an angle of approximately 15 degrees from the surface of the bioprocessing device.

In an embodiment, the low vessel fill level may be less than approximately 50 percent of the vessel's total volume.

In an embodiment, the lower portion of the body may include an attachment mechanism for securing the body to the surface of the bioprocessing device.

In an embodiment, the sensor recess may be formed to allow a first portion of the sensor located within the sensor recess to be rotated so that it can be aligned with and engage a second portion of the sensor that is fixed to the vessel.

According to an aspect of the invention, a tray for receiving a bioprocessing vessel and for attachment to a rocking platform of a bioprocessing device, the tray includes a first end and a second end opposite the first end, an angled portion adjacent to the first end of the tray, the angled portion having a sensor recess that is configured to receive a sensor so that the sensor is at least partially recessed within the angled portion; and the angled portion allows for retention of liquid in the vessel in proximity to the sensor while the platform is rocking back and forth about an axis thereby facilitating use of the sensor with low vessel fill levels.

In an embodiment, the angled surface may be at an angle of approximately 15 degrees from the surface of the bioprocessing device.

In an embodiment, the low vessel fill level may be less than approximately 50 percent of the vessel's total volume.

In an embodiment, the sensor recess may be formed to allow a first portion of the sensor within the sensor recess to be rotated so that it can be aligned with and engage a second portion of the sensor that is fixed to the vessel.

According to another aspect of the invention, a method of reducing a minimum vessel fill level to facilitate use of a sensor includes placing a body on a surface of a bioprocessing device, the body having upper and lower portions, the body extending across substantially an entire width of a vessel; connecting a first portion of a sensor, located in a sensor recess formed in the upper portion of the body, to a second portion of the sensor that is fixed to the vessel; and wherein the body is situated between the surface of the bioprocessing device and the vessel, the body allowing for retention of liquid in the vessel in proximity to the sensor, facilitating use of the sensor with low vessel fill levels.

In an embodiment, the method may further include adding a fluid to the vessel; and agitating the fluid in the vessel by rocking the surface of the bioprocessing device about an axis to perform a bioprocessing procedure.

In an embodiment, the sensor may be a viable cell density sensor and the method further includes measuring viable cell density within the fluid using the viable cell density sensor.

Features which are described in the context of separate aspects and embodiments of the invention may be used together and/or be interchangeable. Similarly, features described in the context of a single embodiment may also be provided separately or in any suitable sub-combination.

### BRIEF DESCRIPTION OF THE FIGURES

The present invention will be better understood from reading the following description of non-limiting embodiments, with reference to the attached drawings, wherein below:

FIG. 1 is a perspective view of a rocking platform bioreactor system suitable for use with embodiments of the present invention;

FIG. 2 is a side view of the system of FIG. 1 depicting a platform and attached tray tilted at an angle that would result from the system's rocking motion;

FIG. 3 is an illustration of a bioreactor bag with a VCD sensor incorporated into the bag;

FIG. 4 is an illustration of a VCD sensor transducer that, in use, is mated to the sensor of FIG. 3.

FIG. 5 is a simplified schematic of a rocking platform bioreactor depicting a mated VCD sensor and transducer and an island effect created by the same.

FIG. 6 is a perspective illustration of an apparatus for facilitating use of a sensor with a vessel, e.g., the bioreactor bag and sensors of FIGS. 3 and 4, according to an embodiment of the invention.

FIG. 7 is a view of the apparatus of FIG. 6 placed on a tray of a rocking platform bioreactor.

FIG. 8 is a top view of the apparatus of FIG. 7.

FIG. 9 is a sectioned illustration of the apparatus of FIG. 6 and a rocking platform depicting the platform in a first angled position.

FIG. 10 is a sectioned illustration of the apparatus of FIG. 9 depicting the platform in a second angled position, opposite the first position.

FIG. 11 is a perspective illustration of an apparatus for facilitating use of a sensor with a vessel, e.g., the bioreactor bag and sensors of FIGS. 3 and 4, according to another embodiment of the invention.

FIG. 12 is a top of the apparatus of FIG. 11 placed on a tray of a rocking platform bioreactor.

FIG. 13 is an additional view of the apparatus of FIG. 12.

FIGS. 14A-14E are graphs illustrating the results of testing to quantify the percentage of VCD data loss associated with use of the apparatus embodiments of FIGS. 6 and 11 at various vessel fill levels.

FIG. 15 is a chart summarizing the data obtained from testing illustrated in FIGS. 14A-14E.

FIG. 16 is a simplified schematic of a rocking platform bioreactor and apparatus for facilitating use of a sensor with a vessel according to an alternative embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible, single-use flexible bags, as well as other containers or conduits commonly used in biological or biochemical processing, including, for example, cell culture/purification systems, fermentation systems, mixing systems, media/buffer preparation systems, and filtration/purification systems.

As used herein, the term “bag” means a flexible or semi-rigid container or vessel used, for example, as a bioreactor or mixer for the contents within.

As used herein, “platform” refers to a rocking platform i.e., “rocker” bioreactor surface that is configured to receive and support a vessel/bag during a bioprocessing procedure. This term includes, but is not limited to, bioreactor trays. While shown and described in association with specific rocker bioreactor systems that include, for example, trays that are affixed or mounted to rocking platforms, the invention is not limited to such systems.

Embodiments may be utilized in connection with a wide variety of biological and chemical processes. Certain embodiments may be utilized in other industries where sensor analysis of a fluid in an environment in which fluid levels fluctuate is desirable. Similarly, embodiments of the invention may also be utilized with sensors other than permittivity based VCD sensors. For example, embodiments may be suitable for use with capacitance or optical VCD sensors, or with non-VCD sensors.

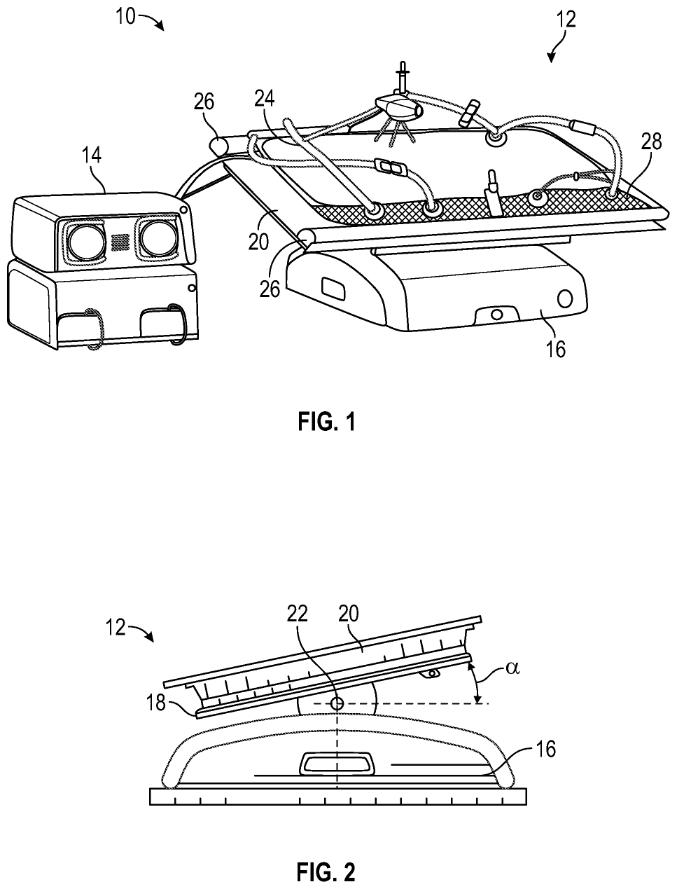

Referring to FIG. 1, an exemplary rocking platform or rocker bioreactor system 10 suitable for use with embodiments of the invention is depicted. As shown, the system 10 includes a bioreactor 12 which is operatively connected to one or more peristaltic pumps 14. The bioreactor 12 is also operatively connected to a controller, e.g., a local or remote computer that provides process/protocol monitoring and the like via a wired or wireless connection (not shown).

The bioreactor 12 includes a surface, e.g., a removable tray 20, which is configured to selectively receive and support a vessel/bag 24 and move in a rocking manner/motion as described in greater detail below. In embodiments, the tray 20 may include a removable lid (not shown) having a hinged door that can be raised to gain access to the tray 20, and any vessel 24 secured thereto.

Referring now to FIG. 2, the bioreactor 12 includes a base 16 that is connected to a rocking platform 18, which, in turn, is connected to the tray 20. The base 16 houses a pivoting mechanism, for example, a motor, which enables the rocking platform 18 to rock back and forth about an axis 22.

The tray 20 may include one or more heating elements, e.g., a heat plate 32, and may also include temperature sensors, so that the contents of a vessel/bag 24 may be accurately temperature controlled.

In use, a vessel/bag 24 is secured to the tray 20 via one or more attachment mechanisms 26, which, in an embodiment, are selectively lockable clips located at opposite ends of the tray 20. The rocking platform and tray 20 may then pivot about the axis 22 creating a rocking motion to facilitate bioprocessing of fluid 28 in the vessel 24.

While rocking, the platform 18 forms an angle α to horizontal at the end point of its upward path, and the rear portion of the rocking platform 18 moves back and forth from an angle of −α to +α. In embodiments, angle ox may be varied by a user and is in a range of about 2° to about 12°. As will be appreciated, however, embodiments of the invention are suitable for use with reactor platforms that move at angles outside of this range.

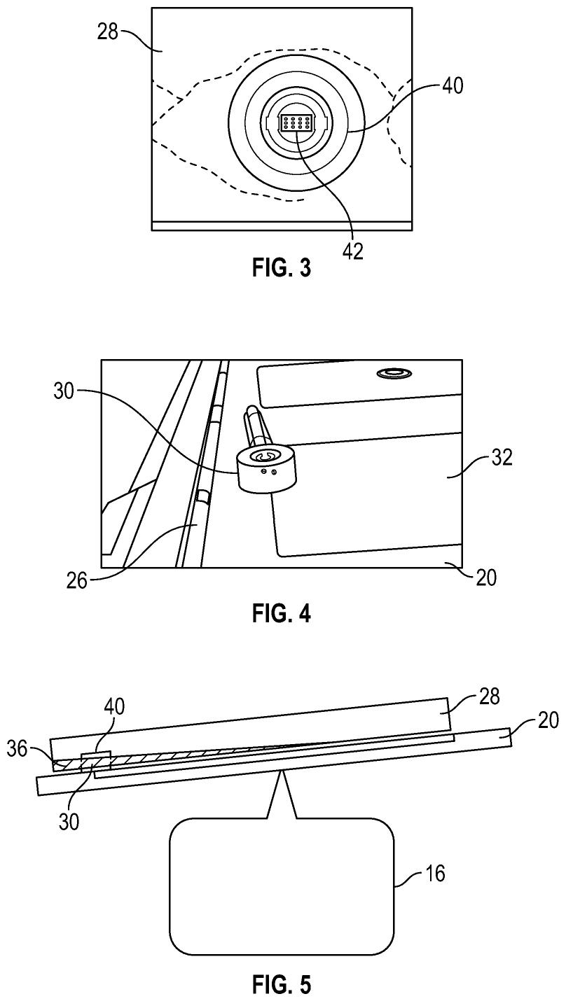

Referring now to FIGS. 3 and 4, in bioprocessing and other contexts, it is desirable to assess viable cell density to evaluate, for example, cell growth. This may be accomplished using a VCD sensor that measures cell permittivity. VCD sensors are typically an assembly of several components which may include a first portion, e.g., a transducer 30, which is mated or connected to a second portion, e.g., a sensor patch 40 that is fixed to a vessel 28.

In embodiments, the sensor patch 40 may be fixed to the vessel 28 via welding (e.g., thermal), or may be otherwise formed in a flexible wall of the vessel 28. In specific embodiments, the sensor patch 40 is welded to a bottom surface at an end of the vessel 28, so that an engagement portion 42, e.g., a pin connector, faces downward so that the sensor patch 40 may selectively engage the transducer 30. As will be appreciated, the position of the sensor patch 40 may vary, and embodiments are not limited to use with sensors in any particular vessel location.

As shown in FIGS. 4 and 5, the transducer 30 has a relatively tall profile/height, however, such that when it is placed on a tray 20 of a rocking platform bioreactor, it extends upward into the vessel 28 raising the height of the sensor patch 40. This extension creates an island effect, where, during the rocking motion of the reactor, the sensor patch 40 may have very little fluid 36 above it or may be raised above the fluid level in the vessel 28. As will be appreciated, when the sensor patch 40 does not have sufficient fluid 36 above it, it cannot obtain an accurate VCD measurement. The island effect is particularly acute at relatively low vessel fill levels and when the rocking platform is tilted away from the sensor patch 40.

Increasing vessel fill levels to address the island effect is often impracticable as the required minimum fill levels would approach maximum capacity levels for many existing vessels. For example, a potentially suitable minimum fill level for a 2 L vessel would rise from 100 ml to over 600 ml.

Placing the transducer 30 directly on a tray 20 (FIG. 4) may also be undesirable as movement of the unsecured transducer 30 during use may stress the area of the vessel 28 surrounding the sensor patch 40. It is also possible that the height of the transducer 30 and sensor patch 40 may alter the wave motion within the fluid 36 that is generated by the rocking motion of the reactor. With the above considerations in mind, embodiments of the invention provide an apparatus and methods for facilitating use of VCD and other sensors with vessels, particularly with low vessel fill levels, e.g., fill levels less than approximately 50 percent of the vessel's total volume.

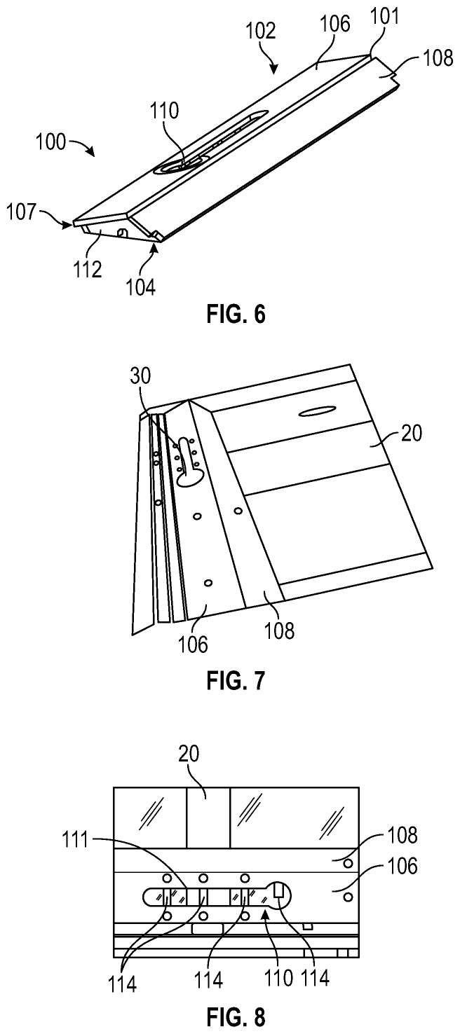

Referring now to FIGS. 6-8 an apparatus 100 for facilitating use of a sensor with a vessel is depicted. The apparatus generally includes a body 101 designed for selective placement on a surface of a bioprocessing device. The body 101 includes an upper portion 102 and a lower portion 104, with the upper portion 102 facing upward towards a user and the lower portion 104 facing downward and contacting the tray or other surface of the bioprocessing device.

As shown, the upper portion 102 includes a first angled surface 106 and a second angled surface 108. The body 101 further includes end portions 112 on opposite ends of the body 101. In the depicted embodiment, the body 101 has a substantially hollow or open interior and the lower portion 104 contacts the rocking platform bioreactor via the end portions 112. In other embodiments, the body 101 may be solid or semi-solid and the entire underside/lower portion 104 may be in contact with the rocking platform.

In certain embodiments, the lower portion 104 may include one or more surfaces, e.g., rubber feet, that have a high coefficient of friction to prevent slip/movement of the body 101 on the surface of the bioprocessing device when it is in motion. In other embodiments, the body 101 itself may be manufactured from a material that is resisting to slipping/movement. In yet other embodiments, the weight of the vessel with fluid may be sufficient to prevent slippage/movement of the apparatus 100 during use.

The first angled surface 106 includes a sensor recess 110. The sensor recess 110 is shaped to receive the transducer 30 of a VCD sensor. In the depicted embodiment, the sensor recess 110 has a substantially keyhole-like shape, which is configured to approximate the shape of the transducer 30, and a depth that approximates the height of the transducer 30, so that the transducer 30 may be at least partially recessed within the sensor recess 110. In embodiments, the transducer 30 may be completely recessed within the sensor recess 110, such that, when mated, the sensor patch 40 of the vessel 28 is substantially flush with the first angled surface 106.

While the sensor recess 110 is depicted having a keyhole shape, the shape and size of the sensor recess 110 may vary depending on the shape, size, and/or type of sensor that the apparatus 100 is to be utilized with. In certain embodiments, the first angled surface 106 may include a plurality of sensor recesses or the sensor recess 110 may be configured to accommodate multiple sensors, e.g., multiple VCD sensors or sensors of differing types.

In certain embodiments, the sensor recess 110 may be configured/shaped to allow the transducer 30 to rotate a small amount so that it may properly mate with the sensor patch 40 without the patch having to rotate potentially stressing the vessel wall to which it is welded/otherwise attached. In a specific embodiment, a tail portion 111 (FIG. 8) of the keyhole shaped sensor recess 110 may be flared outward towards the end of the recess to allow for a +/−5 degree of rotation of the transducer 30 so that the round head portion of the transducer 30 can properly align with the pinned connection portion 42 of the sensor patch 40.

In other embodiments, the sensor recess 110 may also include electronics, e.g., Bluetooth, power supply, and electronics to map/convert communication protocol data, such that no external/bench top module is necessary to power, communicate with, and/or control the VCD sensor. As will be appreciated, the size and shape of the sensor recess 110 may vary depending upon the size and shape of any such electronic components. In certain embodiments, the body 101 may include one or more ports to allow USB connectivity and the like.

Referring to FIG. 8, in an embodiment in which the body 101 is substantially hollow or open, the recess 110 may include one or more brackets 114 which define the depth of the recess 110 and hold the sensor at that depth when it is received in the recess 110. As will be appreciated, in other embodiments, the recess 110 may be formed in/molded into a solid body such that the one or more brackets 114 are unnecessary.

As shown in FIGS. 6 and 10, embodiments of the body 101 may also include an edge portion 107 that is configured to abut a surface of a tray 20 or similar structure on a rocking platform or other surface of a bioprocessing device. In embodiments, this edge portion 107 is angled or pitched to correspond to an angle of a front facing edge or lip of the tray 20 which retains the vessel 28 on the platform during use. As will be appreciated, the size and shape (and presence of) the edge portion 107 may vary and embodiments are not limited in this regard.

While the dimensions of the apparatus 100 may vary, in an embodiment, the body 101 extends substantially an entire width of the vessel that the apparatus 100 is to be used with. The depth that the apparatus 100 extends from front to back on the surface of the bioprocessing device may also vary. However, it may be undesirable for the apparatus 100 to extend onto a heating element (e.g., heating plate) on the surface, such that the transfer of heat from the heating element into fluid in the vessel is significantly inhibited. In certain embodiments, the apparatus 100 may be, at least in part, manufactured from a material that is non-insulative or thermally conductive to address this potential issue. In other embodiments, the apparatus 100 may include a heating element itself.

The overall height of the apparatus 100 may also vary but should be sufficient to allow for a sensor to be recessed within a sensor recess formed therein to address the aforementioned island effect.

In a specific embodiment, the body 101 has a width of approximately 66.5 cm and a height of approximately 3.45 cm. The first angled surface 106 has a depth, meaning the distance the body 101 extends (from front to back) on the surface of the bioprocessing device, of approximately 7.4 cm and the second angled surface 108 has a depth of 5.1 cm. As mentioned, these dimensions may vary and the invention is not limited in this regard.

In specific embodiments, the apparatus 100 may be manufactured from a thermoplastic elastomer such as high-density polyethylene (HDPE). While HDPE may be suitable given its high strength to density ratio, other materials may be employed without departing from the invention. In certain embodiments, the apparatus 100 may be manufactured from nylon, polyurethane, or acrylonitrile butadiene styrene. The apparatus 100 may be vacuum cast, molded, or additively manufactured.

As will be appreciated, other materials may be used to manufacture the apparatus 100. Likewise, other manufacturing techniques may be employed without departing from the scope of the invention.

In embodiments, the apparatus 100 may be an assembly of multiple panels which form, for example, the first and second angled surfaces 106, 108 and end portions 112. The panels may be joined together mechanically and/or chemically via adhesives. In other embodiments, the apparatus 100 may be unitary and/or formed or molded from a single material.

Referring now to FIGS. 6, 9 and 10, a body 101, VCD sensor 30, 40, tray 20, and a vessel 28 are depicted in a sectioned view. As shown, in the depicted embodiment, the apparatus 100 has a substantially triangular profile (e.g., side/end profile), or cross-section, which features an obtuse angle a between the first angled surface 106 and second angled surface 108. The apparatus 100 is substantially triangular in that it has three primary perimeter sides and/or corners. In embodiments, the angle a is approximately 130°. Additionally, the first angled surface 106 is at an angle b of approximately 15° from a surface of the bioprocessing device, e.g., tray 20. As will be appreciated, that angles may vary without departing from the scope of the invention, however, the angle b should be greater than the maximum platform tilt angle, which, in embodiments is 12°.

Embodiments of the invention need not have a substantially triangular profile or cross-section and, it may be possible that the body 101 has an alternate shape such as a chevron (without end portions 112), a rectangular or quadrilateral, or a rounded/domed profile or cross section or other shape entirely.

As will be appreciated, the body 101 has surfaces or edges, e.g., the point at which the first angled surface 106 and second angled surface 108 meet, that are in contact with the flexible vessel 28 during use. In embodiments, these surfaces/edges may be shaped or otherwise designed to minimize stress loading on the flexible material of the vessel 28.

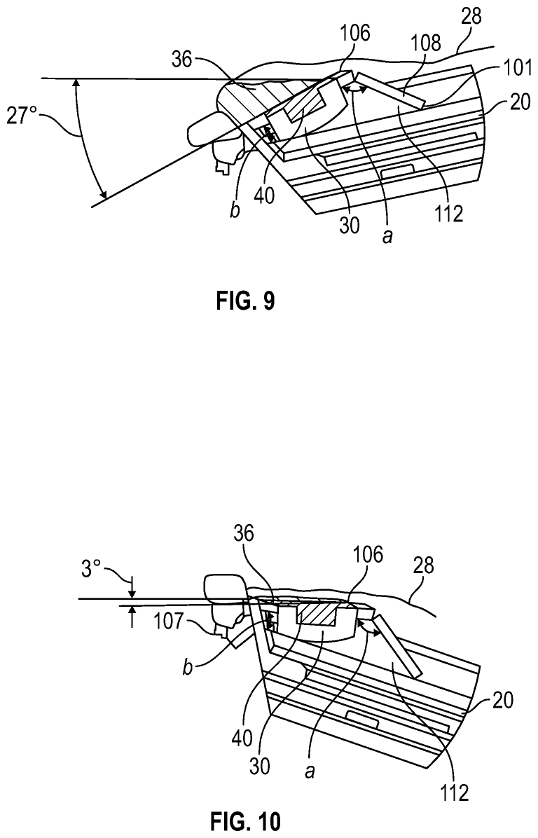

Referring again to FIG. 9, the apparatus 100 and tray 20 are depicted in a first position in which the vessel 28 has been tilted to its maximum angle of 12° towards the sensor patch 40. The vessel 28 is depicted with fluid 36 that is at an approximate 10% fill level (e.g., approximately 5% of the total vessel volume). As shown, the body 101 is situated between the surface of the bioprocessing device, e.g., the tray 20 and the vessel 28. The first angled surface 106 faces a first end, e.g., the front, of the bioprocessing device (not shown). The second angled surface 108 faces a second end, e.g., the back or rear, of the bioprocessing device that is opposite the first end.

As depicted, at a minimal 10% fill level, when the apparatus 100 and tray 20 are in the first position and tilted to their maximum angle of 12° towards the sensor patch 40, the sensor patch 40 is submerged in the fluid 36 in the vessel 28. In this first position, the angle between the sensor patch 40/first angled surface 106 and the fluid fill level is approximately 27°, and the angle b is approximately 15° from the tray 20.

Indeed, as shown in FIG. 10, even in the second position, where the vessel is tilted 12° away from the sensor patch 40, fluid remains pooled over the sensor patch 40 such that VCD measurements may be obtained. In the second position, the apparatus 100 creates a tidal pool like effect. In this position, the angle between the fill level of the fluid 36 and the sensor patch 40/first angled surface 106 is approximately 3°.

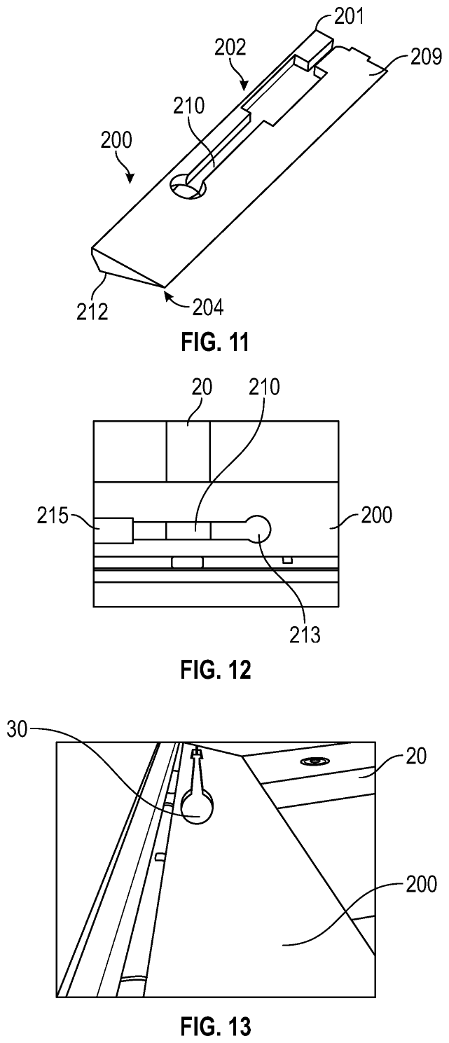

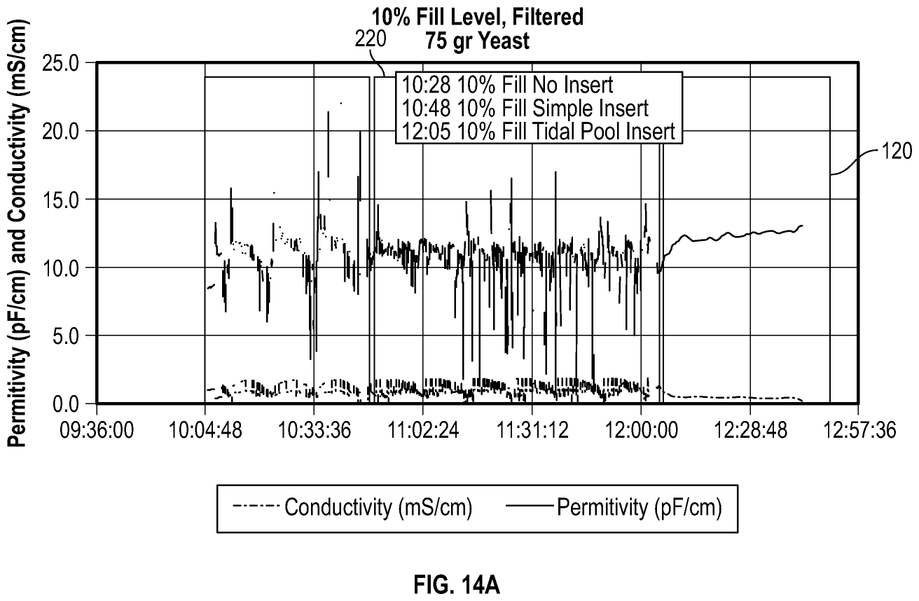

Referring now to FIGS. 11-13, an alternative embodiment of the inventive apparatus 200 is depicted. In this embodiment, the body 201 has an upper portion 202, a lower portion 204, and end portions 212. The upper portion 202 has an angled surface 209 which includes a sensor recess 210. As shown, the sensor recess 210 includes a keyhole shaped portion 213 that is sized and shaped to receive a transducer 30 (or other sensor/sensor component) such that it is at least partially recessed therein, as well as a portion 215 configured to receive electronics.

The apparatus 200 may be manufactured from a variety of materials and may have varying shapes, sizes and angles as discussed in connection with the aforementioned embodiment.

In this embodiment, however, the angled surface 209 faces away from the first end of the bioprocessing device surface, e.g., tray 20, upon which the apparatus 200 is placed. The body 201 has a profile (e.g., side or end profile) or cross-section of a substantially right triangle, with the longest side being the angled surface 209 having the sensor recess 210. In an embodiment, the angled surface 209 is at an approximate angle of 15° from a surface of the bioprocess device (e.g., tray 20) on which is it placed. The apparatus may also have angled edge portion or surface, e.g., the shortest side of the substantially triangle shaped apparatus 200 (FIG. 11). This side may be angled to correspond to an angle of a front lip or edge of the tray 20 or other surface of the bioprocessing device.

As shown, the apparatus 200 is substantially wedge shaped and does not create a tidal pool like effect around the sensor patch 40. The apparatus does, however, mitigate the island effect described herein by allowing retention of liquid in the vessel in proximity to the VCD sensor.

In certain embodiments, the angled surface 106, 209 may be integrated into the surface of the bioprocessing device. For example, a tray 20 may include an angled portion, e.g., an angled surface 106, 209, that has a sensor recess. In an embodiment, the angled portion is adjacent to the first end (e.g., front) of the tray, and is facing the second end (e.g., rear or back) of the tray to allow for retention of liquid in the vessel in proximity to the sensor which the platform is rocking. In other embodiments, the angled surface may face the first end/front of the tray.

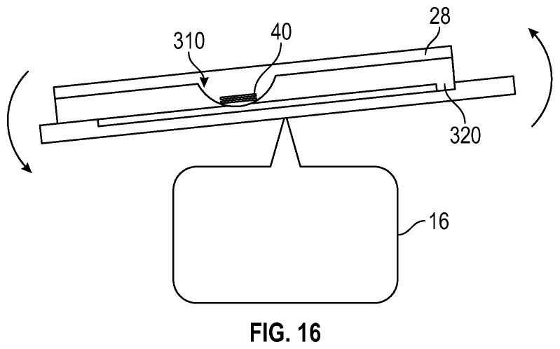

In certain other embodiments, such as that shown in FIG. 16, a tray 320 may not include an angled surface as described herein, rather the tray 320 itself may include a sensor recess/well 310 located at an approximate mid-point of the of the tray 320 on an upper portion 322 of the tray 320. The well 310 is relatively deep and would allow the sensor patch 40 and transducer 30 to be immersed in liquid 28 at nearly all times during the rocking motion, even with low fill levels.

In such embodiments, the tray 320 would be selectively placed on a rocking platform of the reactor. As such, the tray 320 would function as the apparatus body described herein, and the rocking platform would be the surface of the bioprocessing device that the body is selectively placed.

In such embodiments, the well 310 would be sized and shaped to accommodate and recess a sensor of interest, e.g., VCD sensor or otherwise.

Embodiments of the invention also contemplate a method of reducing a minimum vessel fill level to facilitate use of a sensor. The method includes an initial step of placing a body 101, 201 of an apparatus 100, 200 on a surface of a bioprocessing device, such as a tray 20, the body 101, 201 having an upper portion 102, 202 and a lower portion 104, 204, the body 101, 201 extending across substantially an entire width of a vessel 28.

Once placed on the tray, the method includes connecting a first portion of a sensor, e.g., a VCD transducer 30 located in a sensor recess 110, 210 formed in the upper portion 102, 202 of the body, to a second portion of the sensor (e.g., sensor patch 40) that is fixed to the vessel 28. The body 101, 201 is situated between the surface of the bioprocessing device (tray 20) and the vessel 28, the body 101, 201 allowing for retention of liquid in the vessel 28 in proximity to the sensor, facilitating use of the sensor with low vessel fill levels.

In embodiments, the method may further includes adding a fluid to the vessel and agitating the fluid in the vessel by rocking the surface (e.g., tray 20) of the bioprocessing device about an axis to perform a bioprocessing procedure.

In embodiments, the sensor is a viable cell density sensor and the method further comprises measuring viable cell density within the fluid 28 using the viable cell density sensor.

In certain embodiments, multiple apparatus can be used in a single bioreactor.

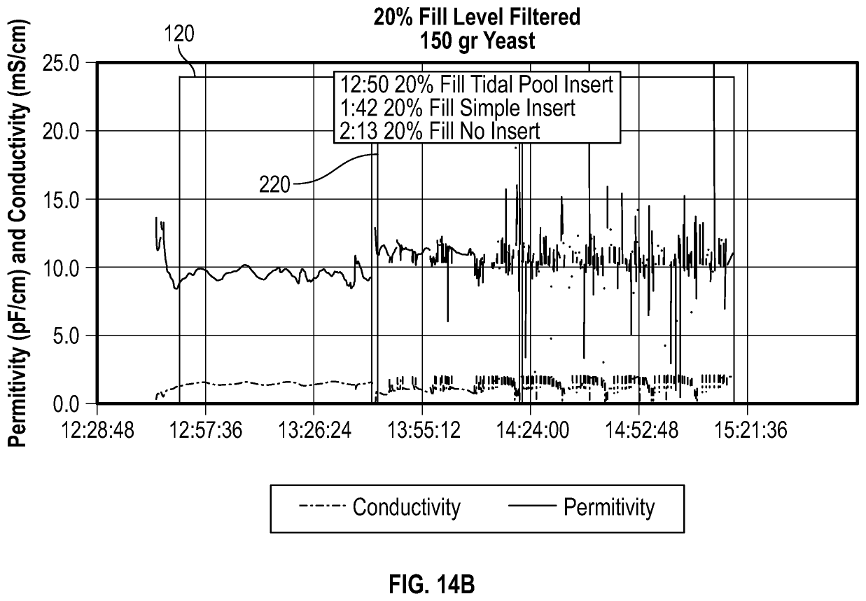

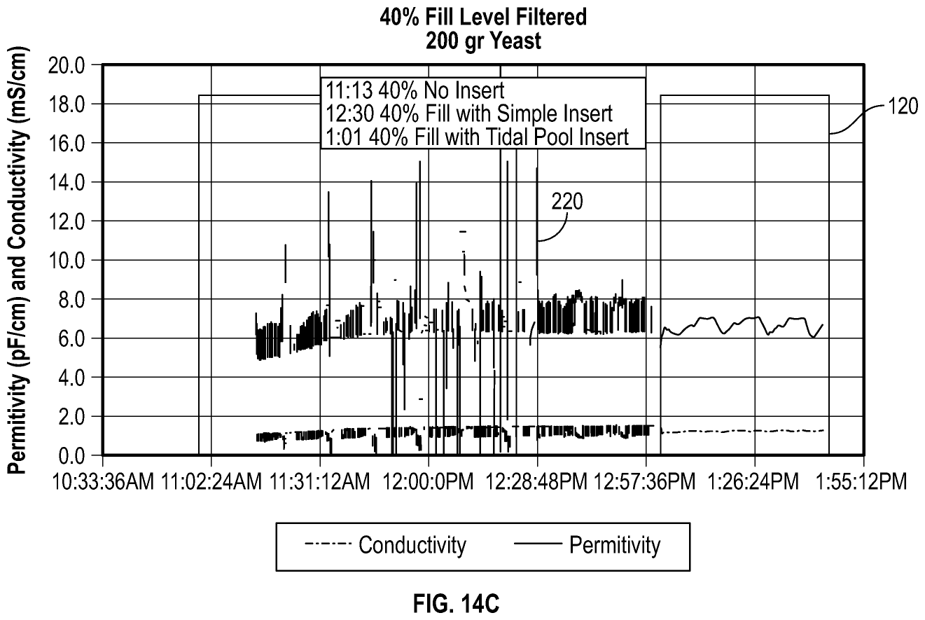

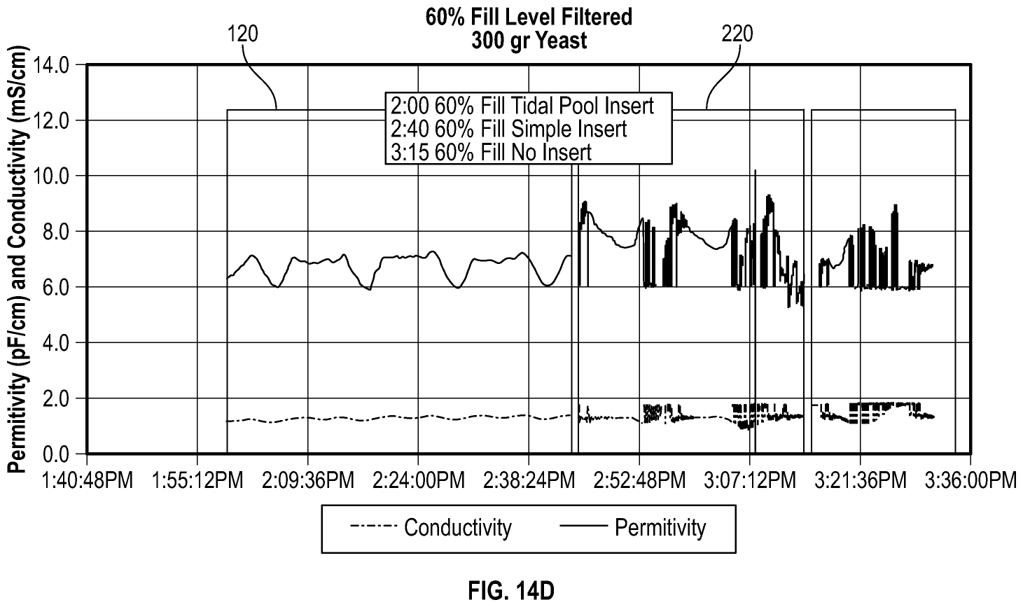

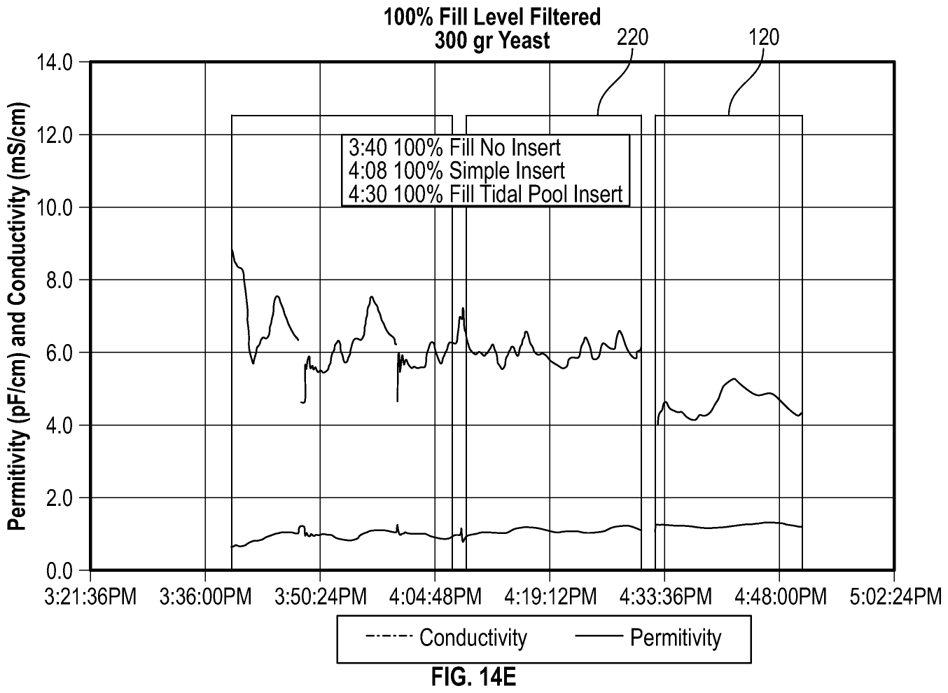

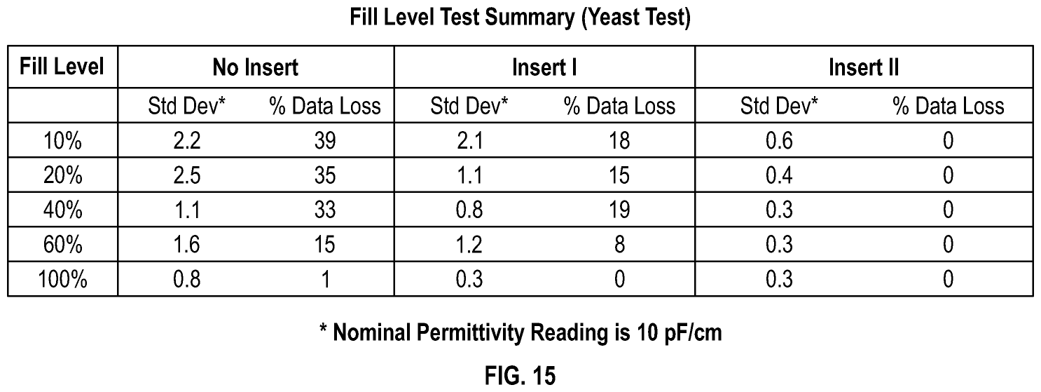

Turning now to FIGS. 14A-14E and FIG. 15, VCD testing was performed using a permittivity based VCD sensor to assess the efficacy of embodiments of the inventive apparatus. In particular, yeast tests were conducted using a rocking platform bioreactor at vessel fill levels of 10% (75 gr yeast), 20% (150 gr yeast), 40% (200 gr yeast), 60% (300 gr yeast), and 100% (300 gr yeast). For each fill level, the test was run with no apparatus on the bioreactor tray, the “tidal pool” apparatus 100, and the “wedge” apparatus 200 described herein. Permittivity as well as conductivity were measured. The testing obtained (and the figures present) raw data without the use of any data filtering.

As shown in FIGS. 14A-14E at each fill level, the results for apparatus 100 and 200 (data attributable to the apparatus are labeled 120 and 220 respectively) are superior to those obtained with no apparatus. In particular, with apparatus 100, there are no breaks in the data 120 representing substantially continuous VCD measurements with little to no data loss while the reactor was rocking.

FIG. 15 summarizes the data from FIGS. 14A-14E into a table. As shown, the “Insert II”, which represents the apparatus 100, has no data loss. Insert I, which represents apparatus 200, has some data loss but not nearly as much as VCD testing where no apparatus was utilized.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

What is claimed is:

1. An apparatus for facilitating use of a sensor with a vessel comprising:
a body having an upper portion and a lower portion, the body extending across substantially an entire width of the vessel, the body configured for selective placement on a surface of a bioprocessing device, the body comprising:
a sensor recess formed in the upper portion of the body, the sensor recess configured to receive a sensor so that the sensor is at least partially recessed within the body;
wherein, in use, the body is situated between the surface of the bioprocessing device and the vessel, the body allowing for retention of liquid in the vessel in proximity to the sensor, facilitating use of the sensor with low vessel fill levels.

2. The apparatus of claim 1, wherein the surface of the bioprocessing device is a tray configured for attachment to a platform of the bioprocessing device that is capable of rocking about an axis to facilitate a bioprocessing procedure within the vessel.

3. The apparatus of claim 1, wherein the sensor in the sensor recess is a first portion of a viable cell density sensor that is configured to selectively engage a second portion of a viable cell density sensor that is fixed to the vessel.

4. The apparatus of claim 1, wherein the vessel is a single-use bioreactor bag.

5. The apparatus of claim 1, wherein the upper portion of the body includes a first angled surface and a second angled surface, the first angled surface including the sensor recess;
wherein, in use, the body is located proximate to a first end of the surface of the bioprocessing device with the first angled surface facing the first end of the surface of the bioprocessing device and the second angled surface facing a second end of the surface of the bioprocessing device, opposite the first end.

6. The apparatus of claim 5, wherein the body has a substantially triangular profile or cross-section with a substantially obtuse angle between the first angled surface and the second angled surface.

7. The apparatus of claim 5, wherein the first angled surface is at an angle of approximately 15 degrees from the surface of the bioprocessing device.

8. The apparatus of claim 1, wherein the upper portion of the body includes an angled surface, the angled surface including the sensor recess;
wherein, in use, the body is located proximate to a first end of the surface of the bioprocessing device and the angled surface is facing a second end of the surface of the bioprocessing device, opposite the first end.

9. The apparatus of claim 8, wherein the body has a profile or cross section of a substantially right triangle with a longest side of the triangle being the angled surface having the sensor recess.

10. The apparatus of claim 8, wherein the angled surface is at an angle of approximately 15 degrees from the surface of the bioprocessing device.

11. The apparatus of claim 1, wherein the low vessel fill level is less than approximately 50 percent of a total volume of the vessel.

12. The apparatus of claim 1, wherein the lower portion of the body includes an attachment mechanism for securing the body to the surface of the bioprocessing device.

13. The apparatus of claim 1 wherein the sensor recess is formed to allow a first portion of the sensor located within the sensor recess to be rotated so that it can be aligned with and engage a second portion of the sensor that is fixed to the vessel.

14. A tray for receiving a bioprocessing vessel and for attachment to a rocking platform of a bioprocessing device, the tray comprising:
a first end and a second end opposite the first end; and
an angled portion adjacent to the first end of the tray, the angled portion having a sensor recess that is configured to receive a sensor so that the sensor is at least partially recessed within the angled portion;
wherein the angled portion allows for retention of liquid in the bioprocessing vessel in proximity to the sensor while the rocking platform is rocking back and forth about an axis facilitating use of the sensor with low vessel fill levels.

15. The tray of claim 14 wherein the angled portion is at an angle of approximately 15 degrees from a surface of the bioprocessing device.

16. The tray of claim 14, wherein the low vessel fill level is less than approximately 50 percent of a total volume of the bioprocessing vessel.

17. The tray of claim 14 wherein the sensor recess is formed to allow a first portion of the sensor within the sensor recess to be rotated so that it can be aligned with and engage a second portion of the sensor that is fixed to the bioprocessing vessel.

18. A method of reducing a minimum vessel fill level to facilitate use of a sensor comprising:
placing a body on a surface of a bioprocessing device, the body having an upper portion and a lower portion, the body extending across substantially an entire width of a vessel; and
connecting a first portion of a sensor, located in a sensor recess formed in the upper portion of the body, to a second portion of the sensor that is fixed to the vessel;
wherein the body is situated between the surface of the bioprocessing device and the vessel, the body allowing for retention of liquid in the vessel in proximity to the sensor, facilitating use of the sensor with low vessel fill levels.

19. The method of claim 18 further comprising:
adding a fluid to the vessel; and
agitating the fluid in the vessel by rocking the surface of the bioprocessing device about an axis to perform a bioprocessing procedure.

20. The method of claim 19 wherein the sensor is a viable cell density sensor and the method further comprises measuring viable cell density within the fluid using the viable cell density sensor.
